(19) United States
(12) Patent Application Publication
(10) Pub. No.: US 2023/0130914 A1
(43) Pub. Date: Apr. 27, 2023

(54) SYSTEM AND METHOD FOR PATIENT CARE HANDOFF

(71) Applicant: The Handoff Company, Inc., Los Angeles, CA (US)

(72) Inventors: Vivek Kaliraman, Los Angeles, CA (US); Dayanand Sharma, Houston, TX (US)

(21) Appl. No.: 18/089,455

(22) Filed: Dec. 27, 2022

Related U.S. Application Data
(63) Continuation-in-part of application No. 16/703,800, filed on Dec. 4, 2019.
(60) Provisional application No. 62/775,532, filed on Dec. 5, 2018.

(51) Int. Cl. G16H 40/20; G16H 10/20

(52) U.S. Cl. CPC G16H 40/20; G16H 10/20

(57) ABSTRACT

Systems and methods are described for a patient care handoff. The disclosed embodiments describe, amongst other things, for example a software architecture that may provide an application/s (e.g. SaaS, software product) for providers to use, may comprise an improved handoff solution, and an objective way to measure, track and report the quality of handoffs. The disclosed embodiments may utilize artificial intelligence in various places to improve handoffs.

### CLAIM TO PRIORITY UNDER 35 U.S.C. § 119

The present application for patent claims the benefit of U.S. Nonprovisional application Ser. No. 16/703,800 filed Dec. 4, 2019, entitled, “SYSTEM AND METHOD FOR PATIENT TRANSFER,” which claims priority to U.S. Provisional Application No. 62/775,532 filed on Dec. 5, 2018, both of which are owned by the applicant hereof, and expressly incorporated herein by reference in their entirety.

### FIELD

The disclosed embodiments relate to systems and methods for patient care responsibility transfers commonly referred to as “handoffs.”

### BACKGROUND

The transfer, acceptance, and quality of patient care handoffs, whether internal to an organization or external, amongst individual or teams of providers and caregivers, are areas of concern in the health industry. Patient handoffs are often inadequate, inaccurate, incomplete, untimely, misinterpreted, and a cause of medical errors/mistakes that lead to increased morbidity and mortality rates. Current systems and methods often rely on the individual caregiver's wherewithal to perform a patient handoff using a “paper” exchange, or with a software equivalent of a “paper” exchange, or a combination of paper and software equivalent with no objective way to ensure accuracy or determine the quality of the handoff. Many factors are involved in the communication failure during a patient handoff, including but not limited to, health care provider training and expectations, language barriers, cultural or ethnic considerations, and inadequate, incomplete or nonexistent documentation. For example, a nurse leaving their overnight shift may be rushed, may forget, may not have access to all the relevant information, may be tired, may not know what should be prioritized, or may not have the skill level to know what is important to inform the nurse starting their shift. The incoming nurse may have the same problems, and especially may not know what to ask, etc. Moreover, there is no objective way to measure the quality of the information communicated or exchanged during patient handoffs. Therefore, there is a need in the art for a system and method for patient care handoff.

### SUMMARY

System and method for patient care handoff are described. In an embodiment, an application for patient handoff, is described comprising non-transitory computer readable medium executable code, comprising: an application control module comprising code to control the application; a patient information module comprising code to gather and store patient information; a clinical information module comprising code to gather and store patient clinical information; an engine module comprising code to create and execute rules based on information from the application modules; a tell and ask assist module comprising code to facilitate the handoff; and a scoring module comprising code to measure the quality of the handoff.

In another embodiment, a method for patient care handoff, is described comprising: determining a patient handoff score (PHS), the patient handoff score (PHS) based on the combination of: determining a numeric value representing a nature of the information exchanged quality (IEQ) for the handoff; determining a numeric value representing an interaction value (INT) between a handoff sender and a receiver; and determining a numeric value representing an efficiency of the handoff.

### BRIEF DESCRIPTION OF THE DRAWINGS

The following embodiments may be better understood by referring to the following figures. The figures are presented for illustration purposes only, and may not be drawn to scale or show every feature, orientation, or detail of the embodiments. They are simplified to help one of skill in the art understand the embodiments readily, and should not be considered limiting.

FIG. 1 illustrates an example of typical breaks in patient care.

FIG. 2 illustrates an exemplary system for patient care handoff in an embodiment.

FIG. 3 illustrates a basic flow of information for facilitating handoffs in an embodiment.

FIG. 4A illustrates a process for a synchronous handoff in an embodiment.

FIG. 4B illustrates a process for a asynchronous handoff in an embodiment.

FIG. 5 illustrates an application for patient care handoff in an embodiment.

FIG. 6A illustrates an exemplary IU that may be created and exchanged for a shift change handoff between outgoing and incoming nurses.

FIG. 6B illustrates an exemplary IU that may be created and exchanged for an interdisciplinary rounding between a nurse and physician.

FIG. 7 illustrates a method for facilitating a handoff in an embodiment.

FIG. 8A illustrates some examples of information triggers in an embodiment.

FIG. 8B illustrates some examples of actions in an embodiment.

FIG. 8C illustrates some examples of handoff process related triggers in an embodiment.

FIG. 9 illustrates a basic process for creating patient IUs for handoff.

FIG. 10A illustrates a method of using AI in an embodiment.

FIG. 10B illustrates another method of using AI in an embodiment.

FIG. 11 illustrates a method for determining a patient handoff score.

FIG. 12 illustrates a method for improving a patient handoff.

FIG. 13 illustrates a system for improving a patient handoff.

### DETAILED DESCRIPTION

Each of the additional features and teachings disclosed below can be utilized separately or in conjunction with other features and teachings to provide a system and method for patient care handoff. Representative examples of the following embodiments, will now be described in further detail with reference to the attached drawings. This detailed description is merely intended to teach a person of ordinary skill in the art details for practicing the preferred aspects of the teachings and is not intended to limit the scope of the embodiments.

In September 2017, the U.S. healthcare accreditation organization—The Joint Commission, issued a Sentinel Event Alert stating, “[f]ailed hand-offs are a longstanding, common problem in health care.” Misalignment of expectations between the senders and receivers during patient handoffs lead to break down of efficient communication needed for successful patient handoff. The Alert also noted that their “database includes reports of inadequate hand-off communication causing adverse events, including wrong-site surgery, delay in treatment, falls, and medication errors,” among many other negative outcomes. Medical errors are the third leading cause of death (greater than 250,000) in the United States. In 2008, medical errors cost the U.S. healthcare system an additional $17.1 billion to provide patient care to manage the consequences of measurable medical errors, such as hospital acquired infections, pressure ulcers, extended length of stay in the hospital, and other errors. The Joint Commission noted in 2012 that up to “an estimated 80% of serious medical errors involve miscommunication between caregivers during the transfer of patient” care and responsibility.

The disclosed systems and methods for patient handoff improve the existing medical field by impacting the outcomes that are attributed to poor or failed patient handoffs/transfers. For example, patient safety may be improved by reducing hospital/healthcare institution acquired conditions. The quality of patient care may be improved, patient length of stay in the hospital may be lowered, improvement in patient satisfaction may increase, improved quality of life and satisfaction scores for clinicians may increase, costs of patient care may be reduced, and/or clinician burnout may be improved. Patient readmission into hospitals may also be reduced as well as patient mortality rates. Providers may be equipped with an objective way to measure the quality of the handoffs, thus, allowing them to improve their processes. Thus, the disclosed embodiments significantly improve the medical field.

The disclosed embodiments for patient care handoffs broadly encompasses a software architecture that may provide an application/s (e.g. software as a service, software product) for providers to use, may comprise an improved handoff solution and an objective way to measure and track the quality of handoffs.

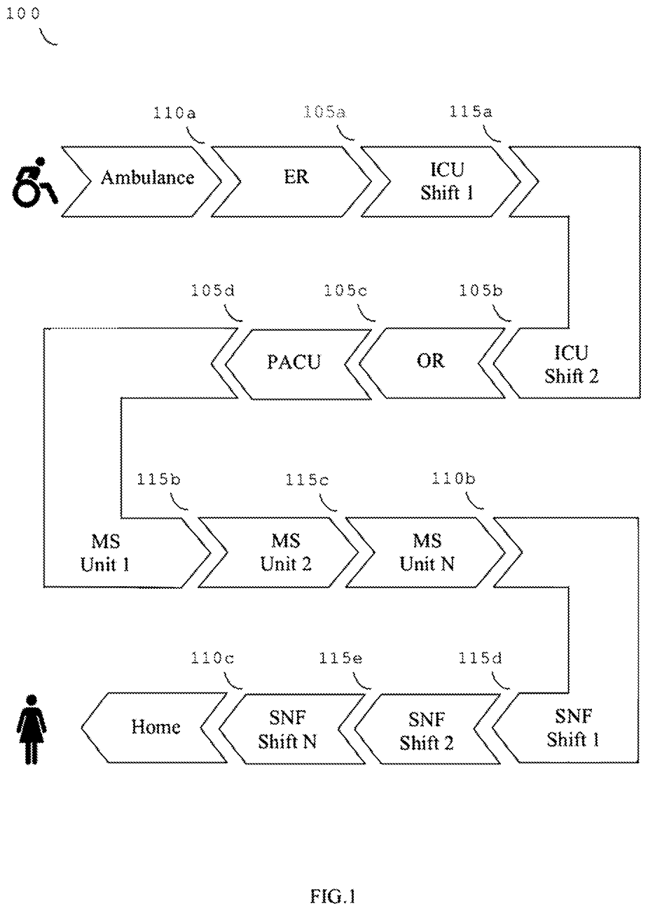

FIG. 1 illustrates an example 100 of typical breaks in patient care. These are examples, and merely used to help describe the types and occurrences of breaks in care. Breaks in patient care may occur at many different places internally within an organization or externally between multiple entities. A handoff may occur during a break or transition in care. A handoff, as used herein, is the process of transferring and acceptance of patient care responsibility. It may encompass both the concepts of patient continuum of care (information exchanges) as well as physical breaks in care (transfers). It may be called different things throughout the industry: handoffs, handovers, sign-outs, sign-overs, turnovers, inter-shift transfers, inter-shift handovers, shift change transfers, patient transfers, transitions of care, transfers of care, service change, substitutions, bedside reports, shift reports, shift-to-shift communications, shift-to-shift reports, discharges, discharge communiqués, discharge summaries, discharge notes, post-operation updates, interdisciplinary transfers, multi-professional handovers, and admissions. It basically involves the process of handing the responsibility of care from one entity to another whether it's intra-entity or inter-entity. An entity may be a physician, nurse, doctor, attending, hospitalist, resident, fellow, patient navigator, respiratory therapist, occupational therapist, physical therapist, case manager, social worker, caregiver, a healthcare personnel, a healthcare team, a department, a hospital, an urgent care center, a skilled nursing facility, nursing home, a clinic, long term care facility, a long term acute care facility, medical office, rehabilitation center or clinic, home health agency, and any entity that transfers or receives the responsibility of patient care. Different types of handoffs may occur. Examples of admission or discharge transfers are shown as 110a-c. For example, a patient may be brought by ambulance to the emergency room (ER) at a hospital 110a. A patient may come into the ER and then be transferred to the ICU department 105a. A patient may be transferred from ICU to an operation room 105b for surgery and then after surgery to post-anesthesia care unit (PACU) 105c and then from PACU to a Medical Surgical (MS) unit 105d. Examples, of inter-unit patient transfers are shown as 105a-d. While in the ICU or MS, the patient care may be transitioned at shift change every 12 hours or 8 hours between nurses 115b, 115c. Examples of shift changes in care are shown as 115a-e. A patient's care may be transferred between nurses leaving shifts 115a-e. Other type of breaks, for example, may be called rounding, shift break, service change, or interdisciplinary. A patient may be discharged from a hospital to a skilled nursing facility (SNF) 110b. A patient may be admitted into the skilled nursing facility 110b. While in the SNF, the patient care may be transitioned at shift change every 12 hours or 8 hours between nurses 115d, 115e. A patient may be discharged from SNF to home 110c.

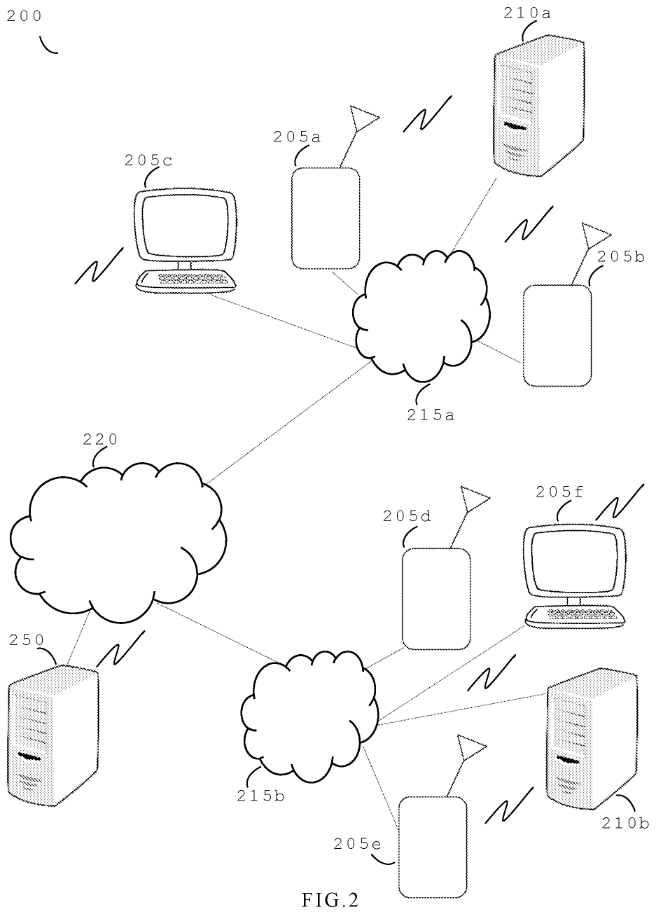

FIG. 2 illustrates an exemplary system 200 for patient care handoff in an embodiment. User end devices such as mobile devices, tablets, smart phones, laptops, desktops, computers, terminals, etc 205a-205n may comprise a user interface for facilitating a handoff and/or a dashboard and may communicate, for example, with the internet 220, LANs 210, WLAN 210, servers, cloud 220, provider servers 210 and each other. Provider servers 210a and 210b may comprise patient data and information and communicate with an application server 250 via the internet/cloud as well as the user devices 205a-205n. Two provider type networks 215a and 215b are shown, but any number of provider networks are envisioned. For example, provider server 210a may be located at a hospital while provider server 210b may be at an oral surgeons office. Networks, servers, databases, applications, users, mobiles, routers, mobile smart devices, laptops, code, PCs, WANs, LANS, security measures, communication protocols, tablets, cloud based computing, high speed networks, software code languages, wireless, and wired transmissions, are all well known in the art. Moreover, the disclosed embodiments may be implemented as a software as a service (SaaS) using any suitable platform. The disclosed embodiments may for example be offered as any suitable tiered subscription model to users. Software as a service (SaaS) architecture is well known in the art. The disclosed embodiments may be able to work with and be implemented on a multiplicity of well-known platforms. The detailed descriptions of which are unnecessary for enabling a POSA to make and use the disclosed embodiments.

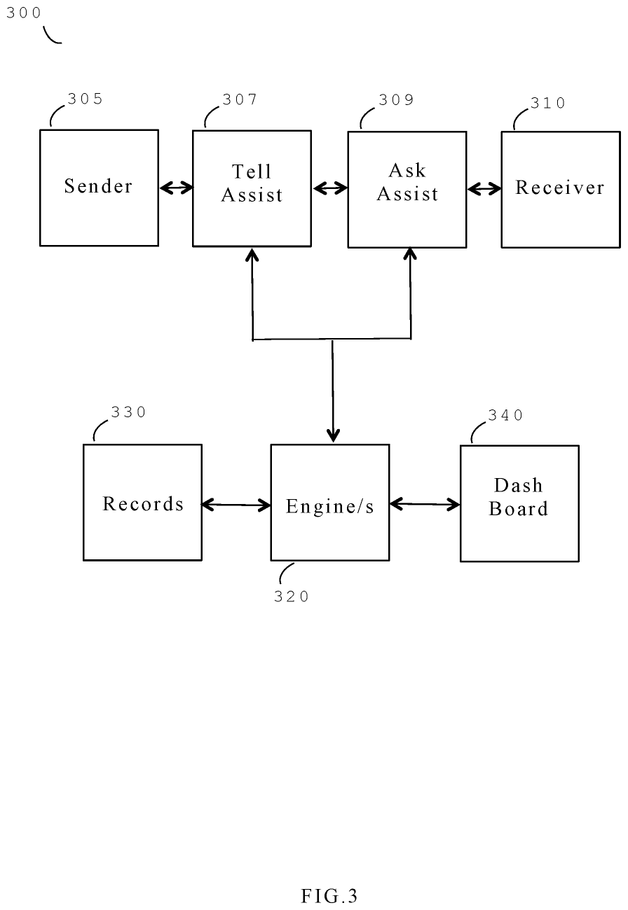

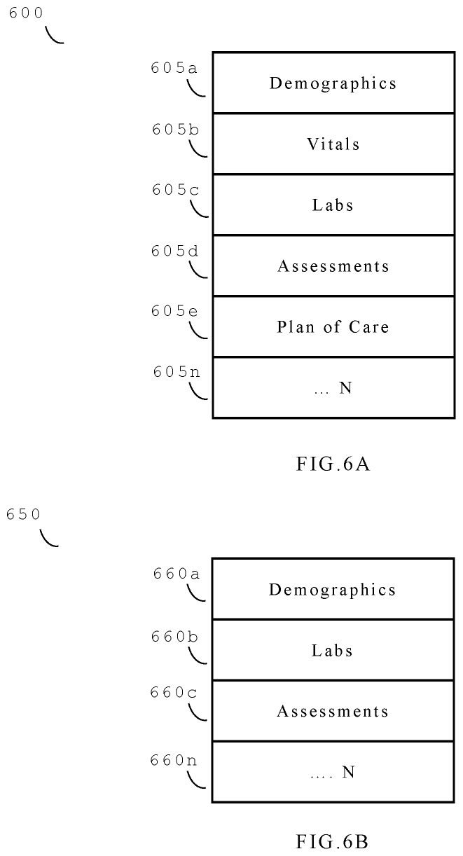

FIG. 3 illustrates a basic flow of information 300 for facilitating handoffs in an embodiment. Information, herein, broadly encompasses: data, numbers, binary numbers, bits, bytes, data bases, analog information, notes, handwritten screen input, text, scanned documents, electronic images, electronic files, media files, PDF files, processed data, raw data, plain text, encrypted data, non-encrypted data, etc. A sender 305 may exchange information with a “tell” assist 307. A sender being the entity who is relinquishing the patient's care. A sender may utilize a device 205a-e as shown in FIG. 2. A “tell” assist 307 may use categorized and prioritized patient information such as situation, background, assessment, and/or recommendation (SBAR). Also for example, the “tell” assist 307 may use categorized patient information prioritized into portions or sections such as Illness severity, Patient summary, Action list, Situation awareness and contingency plan, and/or Synthesis by a receiver or a handoff-receiving clinician (IPASS). A receiver 310 may exchange information with an “ask” assist 309. A receiver being the entity who will be acquiring the patient's care. A receiver may utilize a device 205a-e as shown in FIG. 2. The “ask” assist 309 may use question prompts and discussion points that may be based on the patient's specific information (e.g. diagnosis, condition, unit etc). The ask and tell assists 309 and 307, respectively, may exchange information with each other and/or an engine/s 320. The engine/s 320 may exchange information with records 330 and/or application dashboard 340. The engine/s 320 may use rules to communicate relevant patient information during the “tell” and “ask” assists. In an embodiment, an information unit (IU) as described in more detail below may be generated by the engine/s 320 and shared between the sender 305 and receiver 310. The ask and tell assists 309 and 307, the engines 320 may utilize a server 250 or 210 as shown in FIG. 2. Handoff processes may be synchronous or asynchronous. Synchronous meaning occurring at the substantially same time. Asynchronous meaning occurring at basically different times. Typically, there may be more of a time gap for handoff completion between an asynchronous handoff than a synchronous handoff. Handoffs, of course, may occur in person face-to-face, or across multiple types of media/devices, or a combination of face-to-face and multiple types of media/devices. It may be beneficial for a synchronous handoff to occur face-to-face. Handoffs may occur one-to-one or in batches. For example, an incoming and outgoing nurse handoff during a shift change may process a single patient's handoff between them individually. The incoming and outgoing nurse handoff during a shift change may process different groups of data (e.g. clinical history, lab values, vitals, review of systems, etc.) as an information unit (explained in details in FIG. 6A and 6B) in a single patient's handoff between them individually. While an incoming and outgoing resident during their shift change may process a batch of handoffs (patients #1 . . . #Nth). In an embodiment, the sender 305 and receiver 310 may be using a mobile device, computer, terminal, smart phone, electronic clipboard, or equivalent device, with a user interface that is interactive. User interface(s) displays “cards,” “tiles,” or “slides.” In an embodiment, each “card” may contain an information unit to facilitate the handoff. The user interface(s) may be customized or standard. The user interface may for example, allow a user to invoke and swipe between “cards” or “tiles” and check boxes off or add notes. The user interfaces(s) may facilitate a sequential and structured flow of information between a sender and a receiver during the information exchange at a handoff. The structured flow may be created by dynamically generated checklists that prioritize information depending on multiple variables. For example, some variables such as: the kind of sender or receiver involved (e.g. nurse handoff vs. hospitalist handoff), the type of handoff (e.g. shift change handoff vs. inter-department transfer), the patient's condition (e.g. congestive heart failure vs. hip replacement surgery), the patient's complexity (e.g. patient with congestive heart failure vs patient with congestive heart failure+diabetes), institution or department policies. The user interface(s) may allow feedback or learning information to be entered as well. The sender or receiver may be required to login to the system so that the system may be able to track their handoff information for scoring purposes, and for security reasons as well.

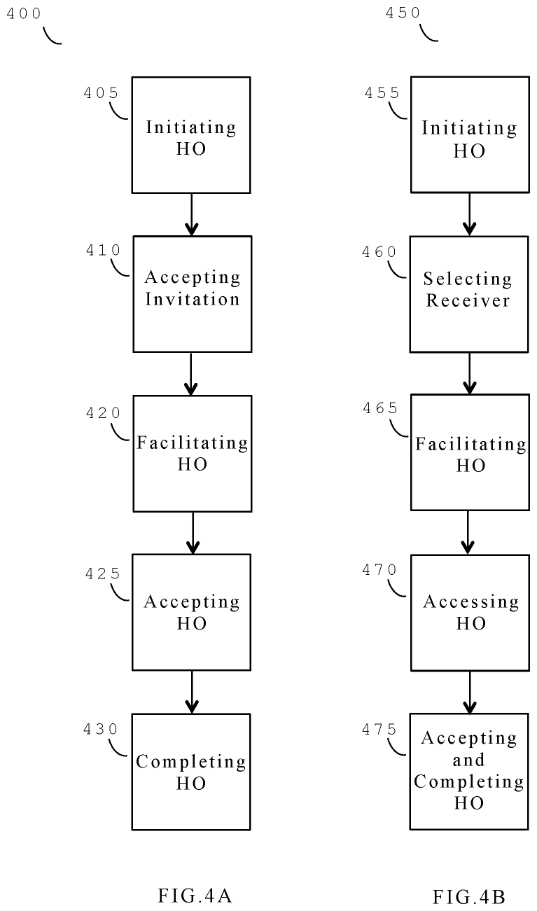

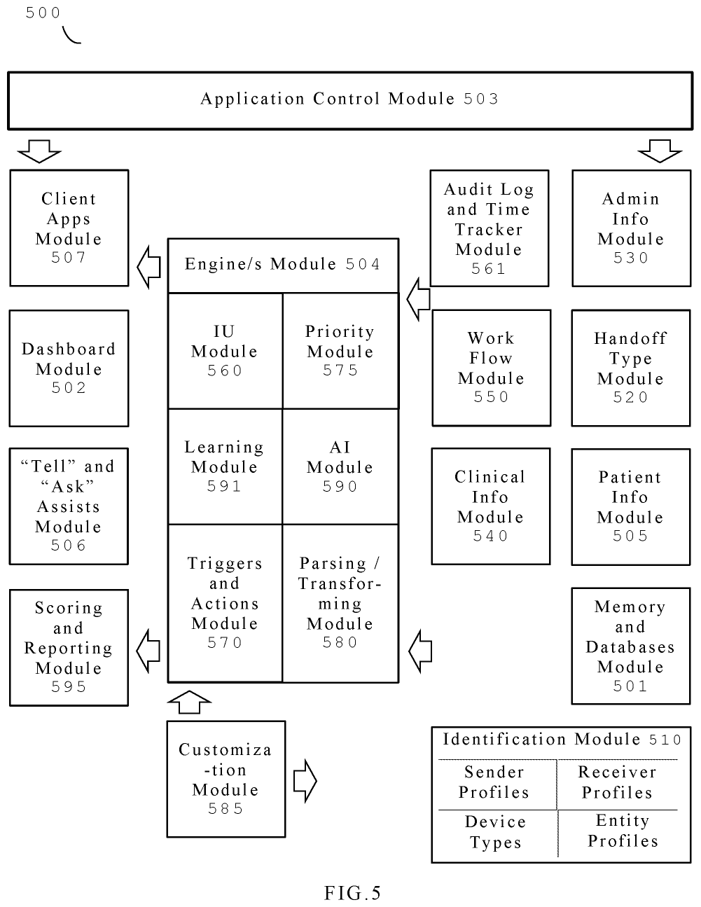

FIG. 4A illustrates a process 400 for a synchronous handoff in an embodiment. At step 405, initiating a handoff. A sender initiates a handoff to a receiver. At step 410, accepting the invitation. A handoff receiver accepts the invitation. At step 420, facilitating the handoff. An application (such as in FIG. 5) may process and control the handoff as described in detail below. The application may help facilitate a handoff by utilizing a tell assist as well as an ask assist. At step 425, accepting the handoff. A receiver accepts the handoff. Then, at step 430, completing the handoff. The handoff sender may complete the handoff.

FIG. 4B illustrates a process 450 for an asynchronous handoff in an embodiment. At step 455, initiating a handoff. A handoff sender initiates a handoff. At step 460, selecting a receiver. The sender selects a handoff receiver. At step 465, facilitating the handoff. An application (such as in FIG. 5) may process and control the handoff as described in detail below. The application may help facilitate a handoff by utilizing a tell assist. The sender may conclude their portion of the handoff. The portion of the patient handoff completed by the sender may be stored waiting for a receiver to retrieve it. At step 470, accessing the handoff. A receiver may access the stored handoff. Then, at step 475, accepting and completing the handoff. The handoff is accepted by the receiver and completed.

FIG. 5 illustrates an application 500 for patient care handoff in an embodiment. The application 500 may comprise various modules for information gathering, information sharing, controlling, and processing in order to facilitate, score and report a handoff. A general direction of data flow is shown by the arrows in FIG. 5, but it is well understood that information may flow differently (e.g. bi-directional) between the modules. The application 500 may comprise a memory and databases module 501 used for gathering and storing information. Medical records and/or patient information may be scattered and residing in different locations/platforms/formats/and systems. Memory and databases module 501 may search, gather, consolidate, and store relevant information. The information may be labeled (e.g. tagged) and placed into databases as such, or the information may be categorized using AI to be “labeled” and placed into a database. Database information may be normalized, separated based on range data or floating numbers, or basically constructed appropriately for the disclose embodiments. Moreover, the information the modules may use, may be simply extracted from other databases. Engine/s module 504 may help to consolidate information for the memory/and databases module 501. The application 500 may comprise an application control module 503 used for processing and controlling the various client applications 507, modules, inputs, outputs, information, displays, dashboards, customizations, analytics, learning, and reporting.

The application 500 may comprise an engine/s module 504. The engine/s module 504 may comprise one or more engines. For example, Engine/s module 504 may comprise a relational database engine, a workflow engine, a rules engine, a fuzzy rules engine, an inference engine, and artificial intelligence (AI) based engines. Information may be shared with the engine/s module 504. The engine/s module 504 may create metadata models and transform data into a different states. Engine/s module 504 using AI may create and modify data models (weights, mapping). Engine/s module 504 may comprise various rules that may be sourced with information from the learning module 591, the customization module 585, the identification module 510, handoff type module 520, the administrative module 530, the clinical information module 540, patient information module 505, the memory and databases module 501, the audit log and time tracker module 561, and the workflow module 550. The engine/s module 504 may determine, modify and create information units (IUs), prioritize information, create and modify triggers and actions, and parse and transform information.

The application 500 may comprise a patient information module 505. The patient information module 505 may gather and store patient information like age, name, address, emergency contact, medical identification numbers, and the like. Patient information may come from any suitable data source. The patient information may be entered directly into the application 500. The patient information module 505 may communicate with and capture external applications or databases of patient information manually or automatically. The patient information may come from information captured may be in one or more electronic medical records (EMR), laboratory information systems, imaging information systems, and the like.

The application 500 may comprise an identification module 510. The identification module 510 may comprise identifying information about the specific senders and receivers. It may also comprise other entities identifying information and even the handoff device type, and locations of an organization, department or unit. The identification module 510 may comprise sender profiles, receiver profiles, device types, and other entity profiles.

The application 500 may comprise a handoff type module 520. The handoff type module 520 may identify what type of handoff it is. The handoff type may matter as to how the engine will process the handoff. The handoff type module 520, for example, may identify a shift change, an inter-unit transfer, a rounding, a service change, a break during shift, an admission or discharge. The handoff type module 520 may identify a synchronous v. asynchronous handoff.

Application 500 may comprise an administration information module 530. The admin information module 530 may comprise information such as insurance enrollment and coverage, payment/invoice/claims information, discharge destination, date of admission, and location information.

Application 500 may comprise a clinical information module 540. Clinical information module 540 may comprise all types of medical related information about the patient. For example, caregiver, entity and provider communications, electronic medical record (EMR), care plans or care provisions, medications, immunizations, diagnostics, lab results, specimens, allergies, codes, isolations, conditions, complications, history, problems, vitals, assessments, procedures, co-morbidities such as multiple diseases affecting a patient, patient acuity, specific patient conditions (e.g. fall risk, on ventilator, etc.), family history, imaging results, and the like.

Application 500 may comprise a workflow module 550. The workflow module 550 may comprise information exchanged during the handoff (e.g. how many IUs where exchanged), supply requests, tasks, orders, appointments, schedules, episodes of care, and encounters. Workflow module 550 may record information regarding types of interactions between clinicians using the disclosed embodiments. In some embodiments, when a sender or receiver taps, clicks, or otherwise enters input identifying specific patient information that is shared during the patient handoff, workflow module 550 may classify the presence or absence of the taps, clicks, or other inputs to a user interface. The user interface (cards, slides, tiles, buttons, clicks or taps etc.) may be displayed in a variety of manners. In an embodiment, colors are used to help distinguish categories of information and tasks etc. When the sender or receiver single clicks, swipes or taps or double clicks or taps (or makes any other varying type of input) on the discussion points for which that user receives a complete or incomplete answer, respectively, workflow module 550 may classify the taps or clicks (e.g., as complete or incomplete answers). Workflow module 550 may also record any other suitable information regarding an interaction between senders and receivers such as, for example, a length of time between a start and a completion of a patient handoff, interruptions occurring during the patient handoff, a sender and/or a receiver identities, and/or any other desired information regarding the patient transfer (e.g., patient handoff). This information may be used to help determine the quality of handoffs.

Application 500 or Engine/s module 504 may comprise a deep learning or Artificial Intelligence (AI) module 590. The deep learning/AI module 590 may be used by the engine/s module 504 for various tasks that may help prioritize patient information, parse and transform information, classify information, create and modify triggers and actions, and create and modify IUs. The AI module 590 may be used to perform natural language processing (NLP) of information that exists as notes or free text or as non-discrete information or as narrative notes in clinical information module 540 or patient information module 505 or information entered into the system. The AI module 590 may also help to more accurately measure the quality of handoffs.

Application 500 or Engine/s module 504 may comprise information unit (IU) module 560. The information unit (IU) module 560 may create, store and comprise basic units (IUs) of information exchanged between the sender and receiver during a handoff. The IUs may consist of previously stored templates that get filled in with information for a handoff, or session created IUs that are based on the patient's specifics, or any combinations thereof. The IUs may be created by the engine/s module 504 to comprise different information per different type of handoff identified (and other factors) from module 520, per different type of clinical information identified (and other factors) from module 540, per different type of patient information identified (and other factors) from module 505, per different type of identification information noted (and other factors) from module 510, or per different type of admin information identified (and other factors) from module 530. The engine/s module 504 may determine what IUs are complied, what information is in each IU, the number of overall information units (IUs) that can be created, and what types of IUs can be shared during synchronous and asynchronous handoffs, etc. Engine/s module 504 may for example, provide a greater number of information per IUs for a synchronous nurse shift change handoff than an asynchronous hospital discharge handoff. The Engine/s module 504 may utilize deep learning or artificial intelligence (AI). For example, AI module 590 may collate different pieces of information in an IU or different number of IUs for different kinds of handoffs and the like.

Application 500 or Engine/s module 504 may comprise a parsing/transforming module 580. The parsing/transforming module 580 may be used to parse, filter, or transform information.

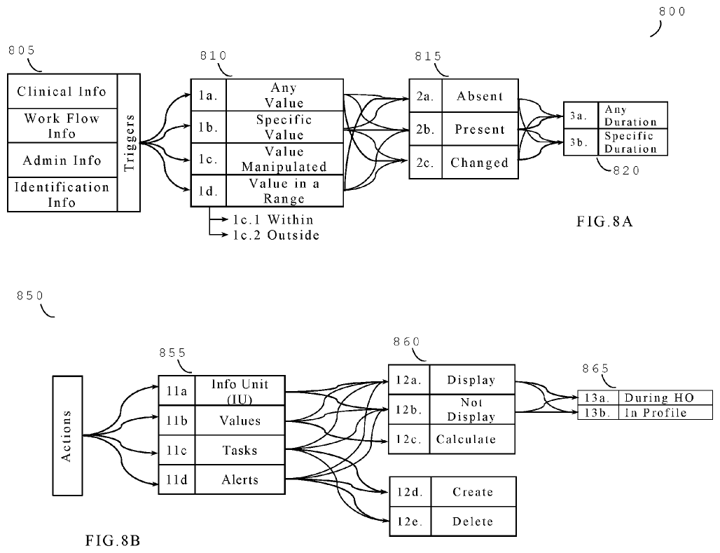

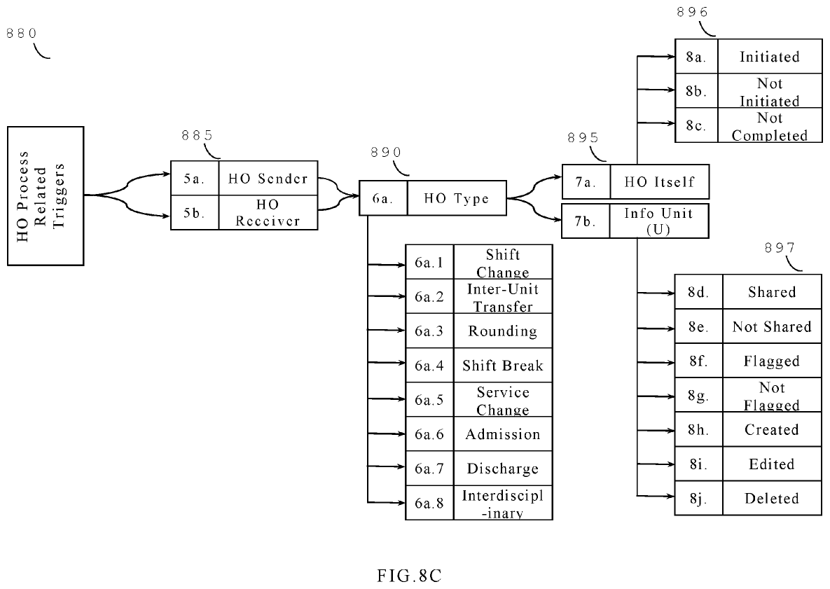

Application 500 or Engine/s module 504 may comprise a triggers/actions module 570. The triggers and actions module 570 may have single triggers or combinations of triggers that lead to designed outcomes for the handoff processes. The triggers may be structured as a hierarchy of triggers. FIG. 8A-C illustrates some examples 800 of information triggers 805, handoff process related triggers 880, and actions 850 in an embodiment. More triggers and actions are envisioned in the embodiments: triggers and actions may be part of a standardized package or be customizable statically or dynamically. For example, AI module 590 may help create triggers and or actions based on learning. A particular hospital may have a set of triggers for one site that isn't included in another site. A hospital may have a set of triggers for one department or unit that isn't included in another department or unit. Many possible permutations for triggers and actions therefore exist. Information triggers 805 may have specific nested sub-triggers associated 810 with them. For example, clinical data 805 may have a specific lab blood test trigger and sub-trigger 810 (1c.) may trigger if the resulting value is outside a normal range (1c.2). Sub-triggers 875 may be status based, for example, the blood lab value outside the normal range may be further defined as a “changed” value (2c.) and further defined 820 by a time duration (3b) (e.g. in the last 4 hours or last 12 hours). As shown in FIG. 8 by the arrows, the triggers, sub-triggers, and actions may be single or combinations. Actions 850 may create events or tasks or things to occur. For example, a sender may initiate a handoff on their mobile device via their user interface. That action may trigger 855 an information unit (IU) to be created (11a). Further parameters 860 may decide to “display” (12a) the IU information. While further sub-parameters 865 may decide to display the IU information only in the patient profile (13b). Handoff process related triggers 880 may be sub-triggered 885 by the sender (5a.) and further sub-triggered 890 by what type of handoff it is, for example, a discharge (6a.7). Other sub-triggers 895 may in turn trigger other sub-triggers 896 and 897 and so forth.

Application 500 may comprise a learning/feedback module 591. The learning/feedback module 591 may provide bias node inputs for AI. The learning module 591 may prompt and gather feedback information from the various entities. For example, it may prompt a head nurse to provide feedback on a completed handoff. The feedback information may be shared as an AI input source. For example, the head nurse may provide input that a certain blood test wasn't exchanged during a handoff but relevant prior to a patient's surgery. The AI can modify the input modeling (weights) using the feedback to optimize patient handoffs for the future.

Application 500 may comprise a dashboard module 502. The dashboard may reside and/or may be displayed on end user devices. For example, a user may login to the dashboard using a smartphone or a workstation in a hospital. The dashboard contents may comprise a user interface that helps display data in a manner that is easier to view and make sense of for the end users. Dashboards may be customized for providers using customization module 585 described below. The dashboard content display may be customized differently for different end users. For example, the nurse might see a specific cluster of information while a physician might see a different cluster of information about patient(s), handoff(s), etc. and a patient himself or herself might see a different cluster of information. Managers may have access to reporting modules while nurses may not.

Application 500 may comprise a scoring/reporting module 595. The scoring/reporting module 595 may for example report numeric scores (e.g., PHS) described below to clinicians, healthcare institutions, and/or any other desired recipient. The reports may be generated at any desired level such as, for example, a patient level, a unit level, a hospital level, a nurse level, and/or any other suitable level (e.g., organizational tier or level or reporting). Details of the handoffs (handoff times, IUs shared) etc may be generated and shared as reports. The scoring and reporting module may track (statistics) the history of the handoffs.

Application 500 may compromise a customization module 585. The customization module 585 may allow for manual and automatic customization. The customization module 585 may, for example, allow a hospital to customize the language of the discussion points, or prompts that are presented to the handoff receiver, customize the information that is allowed to be shared during handoff in different departments and units, or the customization of reports generated for different identities using identification module 510, and the like. The customization module 585 may utilize deep learning or artificial intelligence (AI) module 590 to automatically customize information during handoff process.

Application 500 may comprise a “tell” and “ask” assist module 506. The tell and ask assist module 506 may help facilitate the handoff as described in the embodiments.

Application 500 or Engine/s module 504 may comprise a priority module 575. For example, the engine(s) may determine that some patient, clinical or other related information is more important than other for handoffs. The priority module 575 may utilize deep learning or artificial intelligence (AI) module 590 to prioritize information.

Application 500 may comprise an audit log and time tracker module 561. This module, may be an augment to workflow module 550, or as with any module, may be an integral part of another module (e.g. workflow module 550), and may help track different user actions e.g. viewing and modifying patient information, starting handoff, exchanging information during handoff, finishing handoff, role switching, accessing reports, login and logout etc. The time tracker component of the module may help compute time spent in viewing patient information before starting handoff, time spent in each IU exchange, time spent during handoff.

FIG. 6A illustrates an IU 600 that may be created and exchanged for a shift change handoff between outgoing (e.g. handoff sender) and incoming (e.g. handoff receiver) nurses. IU 600 may comprise different information such as demographics 605a, vitals 605b, labs 605c, assessments 605d, plan of care 605e, and . . . Nth 605n. The IUs may be created manually, automatically, based on AI (learning) or combinations thereof.

FIG. 6.B illustrates an IU 650 that may be created and exchanged for an interdisciplinary rounding between a nurse and physician. IU 650 may comprise different information such as demographics 660a, labs 660b, assessments 660c, and . . . Nth 660n. The IUs exchanged may be exchanged one-to-one between individuals or in batches depending on the nature of the entities involved. For example, a single IU may be exchanged between two incoming and outgoing nurses during a shift change. While a batch of IUs may be exchanged during an outgoing social worker to incoming social worker.

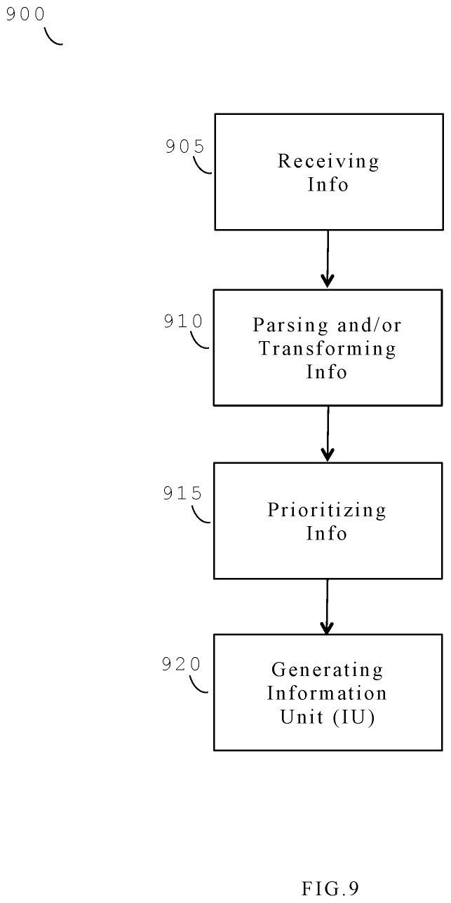

FIG. 9 illustrates a basic process 900 for creating patient IUs for handoff. At 905 receiving information. Information may be received internally or externally, for example, from memory/databases module 501, the information module 505, client applications 507, the clinical information module 540, records 330, or any information relevant to the accuracy and effectiveness of a handoff. At 910, parsing and/or transforming the information. Engine/s module 504 may transform information and restructure it into easily digestible pieces of information. For example, engine/s module 504 may parse (e.g., use algorithms to parse) information and to reconfigure the information. Engine/s module 504 may also use AI module 590 to help parse and transform information NLP and related Artificial Intelligence (AI) to assign clinical meaning and/or context to portions (e.g., different pieces) of information. The parsed information may be structured and appropriately categorized (e.g., into categories such as history of present illness, review of a system, lab value, and/or any other suitable categories). The pieces of parsed information may be given (e.g., assigned) a time stamp and a creator name. For example, the portions of parsed information may be displayed on a “card” or “tile” (e.g., provided virtually on a user interface) that may comprise the information as described for example above. It is also contemplated that the card or slide may be printed. The tile with information may be displayed under different labels or categories (e.g., under any desired category or label). In some embodiments, information regarding tasks that a clinician is scheduled to perform may be transformed into cards or tiles that may be labeled as reminders. The reminders may be provided as alerts by the system (e.g., provided as alerts to a handheld device such as a smartphone or tablet or any other suitable user interface). The “cards” or “slides” may be provided to client applications and/or end user devices such as 507 and 205a-e, and 305, 310.

At 915, prioritizing the information. The prioritization may be driven by multiple factors such as, for example, patient name, unique identifiers, patient age, patient gender, patient diagnosis, a problem list or issue list, patient condition, patient co-morbidities, review of systems, assessments, allergies, code, vitals, patient mobility, labs, imaging results, orders, consults, procedures, contingencies, plan of care, patient acuity, days of hospital stay, transfusion status, department or unit in which the patient is admitted or transferred, discharge disposition (target location for discharge), target for patient transfer, nature of the handoff sender and receiver (e.g. nurse vs hospitalist vs. case manager), type of handoff (e.g. shift change vs. inter-unit transfer vs. rounding) and/or any other suitable factor. A prioritization set (e.g., a set automatically based on an operation of one or more algorithms and/or any desired predetermined criteria) may be overridden by a clinician (e.g., overridden based on a user input). Engine/s module 504 may also use the deep learning/AI module 590 to help prioritize information.

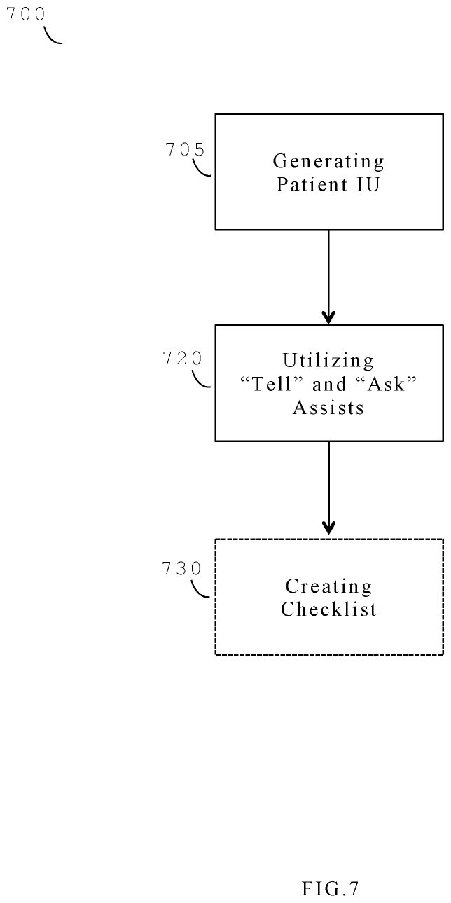

FIG. 7 illustrates a method 700 for facilitating a handoff in an embodiment. At 705, generating at least one patient information units (IU). The IUs may be generated, for example, using the methods as illustrated in FIG. 6A, FIG. 6B, and FIG. 9. The IUs may be previously stored as templates for information to be filled in for the handoff. The IUs may be generated per session based on the patient's specifics. The IUs exchanged may be both a combination of previously stored filled in templates as well as session created IUs. At step 720, utilizing tell and ask assists. Engine/s module 504 may operate to categorize patient information. For example, patient information may be categorized and prioritized into varying sections such as situation, background, assessment, and/or recommendation (SBAR). Also for example, the engine may categorize patient information prioritized into portions or sections such as Illness severity, Patient summary, Action list, Situation awareness and contingency plan, and/or Synthesis by a receiver or a handoff-receiving clinician (IPASS). Engine/s module 504 may also categorize patient information prioritized into any other suitable or desired portions or sections. The exemplary portions or sections may be standardized or customizable for a given hospital, unit, department, handoff type or sender/receiver and/or any other suitable organization or grouping. The tell and ask assists may use IUs created by the Engine/s module 504. The Engine/s module 504 may operate to exchange patient information. In at least some exemplary embodiments, the handoff sender may describe categorized patient information to a handoff receiver by systematically moving from one section to the next (e.g., based on prompting or other output provided by the system and/or input provided by the sender). Also for example, the handoff sender may enter input into the system. For example, the sender may click or tap on each card or tile displayed on a user interface of categorized patient information while and/or after describing a given portion or section of patient information. For example, a given categorized patient information card or slide tile may be displayed (e.g., displayed on a screen of a user interface) of a handoff receiver after the handoff sender has clicked, tapped, or otherwise provided input regarding that tile or card on the sender's user interface. The system may pause (e.g., or be paused by a user) to allow a handoff-receiving clinician (e.g., receiver) to take notes during the exchange of patient information. For example, the system may provide a prompt (e.g., element or button that may be pressed by a receiver) so that a note-taking feature that may be displayed to the receiver may be utilized by the receiver (e.g., and the notes may be later accessed by the receiver using the exemplary system). A receiver may be presented with discussion points by the system (e.g., each discussion point sequentially, for example one at a time). In at least some exemplary embodiments, the handoff-receiving clinician (e.g., receiver) may provide input (e.g., single clicks or single taps) on the discussion points for which the receiver has received a complete answer. The handoff receiver may also provide input on the discussion points for which the receiver has received an incomplete answer. The handoff receiver may input to the system identification of discussion points that resulted in a complete answer to receiver questions, and discussion points that resulted in an incomplete answer to receiver questions. Users may use any technique for providing varying or differentiating input to the exemplary system. In an embodiment, AI may be used to help improve the IUs and Handoff process/information exchanged. For example, a receiver may ask a question and the AI may learn to incorporate the question into future exchanges, etc.

Optionally, at 730, creating checklist. Application 500 or Engine/s module 504 may operate to provide a dynamic checklist. The checklist/s may be dynamically created and comprise items such as discussion points for the handoff-receiving clinician (e.g. receiver) to discuss with the handoff-giving (e.g. sender) clinician. Discussion points may be generated based on predetermined criteria (e.g., specific triggers) regarding parsed or transformed patient information. The exemplary triggers for discussion points (e.g., items to be discussed) may come from various engine modules: clinical information module, workflow module, administrative module, and identification module, etc. They may comprise, for example, patient age, patient gender, diagnosis, problem list or issue list, patient condition, patient co-morbidities, review of systems, allergies, codes, assessments, vitals, mobility, labs, imaging results, orders, consults, procedures, contingencies, plan of care, patient acuity, days of hospital stay, allergies, code, transfusion status, department or unit in which the patient is admitted or transferred, and/or any other suitable discussion points. Discussion points may for example be customizable for multiple triggers (e.g., any desired plurality of triggers) based on user preferences and/or other exemplary operation of the system.

In an embodiment, scoring or determining the quality of patient handoffs may be determined and reported. It may also be used as an input source for AI. Being able to track and objectively determine the quality of handoffs will improve the medical field significantly. Providers may be able to objectively evaluate performances on an individual, unit, department, or organizational level. They may be able to use the scoring/reporting to improve processes, staff departments, and pinpoint areas that need improvement etc. They may be able to keep a statistical (historical) record over time for comparisons. For example, one hospital may have a higher rate of patient care issues than another. The hospital may not be able to figure out where the errors in care are occurring. For example, a handoff in the current art (without the disclosed embodiments) would occur typically between two people using paperwork (or an electronic device equivalent) and there would be no measurable or objective way to determine if that handoff was even adequate. The disclosed embodiments enable providers to objectively measure the handoff quality and track performances. In the example given, the hospital may be able to use the scoring and reporting to learn that an individual or a certain department is providing poor handoffs and use the information to correct and maintain proper procedures. Moreover, the scoring and reporting may be used by the AI module 590 to learn to improve the handoff processes. The quality determination may comprise three main areas of criteria: the nature of the information shared during the exchange, the interaction of the handoff sender and receiver, and the efficiency of the patient handoffs. These three criteria may be used to provide measurement of handoff quality independently and collectively. There may be many different ways to measure or score the quality of a handoff based on the criteria. A collective measurement may be called a Patient Handoff Score (PHS). In an embodiment, a Patient Handoff Score (PHS) may be expressed on a scale from 0-60. It may be determined by the following equation of which will be explained in detail below:

PHS=(IEQ-HS+IEQ-HR)/2×INT×HOLT

An Information Exchange Quality (IEQ) may be measured by the quantity and quality of information shared/exchanged by the Handoff Sender (IEQ-HS) and by the Handoff receiver (IEQ-HR). Recall that an IU is shared during a handoff (e.g. FIG. 6A). The quantity of IUs shared during a patient handoff may be measured as a percent of all IUs shared. Application 500, engine/s module 504, or scoring and reporting module 595 may determine IEQs. An IU quality may be determined by the category in which they are placed or if they are generated by rules specific to the department or specific to the patient. Depending on which category, weights may be provided as follows:
A. IUs determined by the handoff categorization rules (SBAR, IPASS, etc.) are weighted as normal weight (W_{T}=1)
B. IUs determined by the department or Unit level rules are weighted three times more important than handoff categorization rules (W_{T}=3)
C. IUs determined by the rules based on a patient's clinical condition are weighted seven times more important than handoff categorization rules (W_{T}=7)

Table 1-1 helps to illustrate an example for calculating an Information Exchange Quality by a Handoff Sender (IEQ-HS). The weighted average percentages and IEQ-HS scores are shown for three (#1-3) handoffs by a single sender. For the first handoff (#1) by the sender, the total number of IUs available to share is 30 for category specific information units (e.g. SBAR, IPASS). The actual number of category specific IUs shared was 28. The percentage of IUs actually shared is thus (28/30)*100=93.33%. For the first handoff (#1) by the sender, the total number of IUs available to share is 20 for Dept./Unit specific information units. The actual number of Dept./Unit specific IUs shared was 18. The percentage of IUs actually shared is thus (18/20)*100=90%. For the first handoff (#1) by the sender, the total number of IUs available to share is 10 for patient specific information units. The actual number of patient specific IUs shared was 10. The percentage of IUs actually shared is thus (10/10)*100=100%. The average weighted percentages for handoff #1 is equal to [(93.33)(1)+(90.00)(3)+(100)(7)]/11=96.67%. The IEQ may be expressed on a scale of 1-10. The IEQ-HS score in table 1-1 is generated by taking the Weighted Average Percentage and dividing it by 10. So for example, The Weighted Average Percentage of handoff #1 is 96.67%, and 96.67% divided by 10 is 9.667.

TABLE 1-1
Information Exchange Quality by Handoff Sender (IEQ-HS)

| | | | Sender’s HO #1 | | Sender’s HO #2 | | Sender’s HO #3 | |
|---|---|---|---|---|---|---|---|---|
| | | Total # | Total # | | Total # | | Total # | |
| | Weights | of IUs | of IUs | % IUs | of IUs | % IUs | of IUs | % IUs |
| IU Category | (W_{T)} | available | Shared | Shared | Shared | Shared | Shared | Shared |
| A. Category Specific | 1.0 | 30 | 28 | 93.33% | 14 | 46.67% | 7 | 23.33% |
| Information Units | | | | | | | | |
| (e.g. SBAR, IPASS) | | | | | | | | |
| B. Dept./Unit Specific | 3.0 | 20 | 18 | 90.00% | 18 | 90.00% | 18 | 90.00% |
| Information Units | | | | | | | | |
| C. Patient Specific | 7.0 | 10 | 10 | 100.00% | 10 | 100.00% | 10 | 100.00% |
| Information Units | | | | | | | | |
| (e.g. clinic condition) | | | | | | | | |
| Weighted Average | | | | 96.67% | | 92.42% | | 90.30% |
| Percentage | | | | | | | | |
| IEQ-HS | | | | 9.667 | | 9.242 | | 9.030 |

Table 1-2 helps to illustrate an example for calculating an Information Exchange Quality by a Handoff Receiver(IEQ-HR) calculated in the same manner as table 1-1.

TABLE 1-2
Information Exchange Quality by Handoff Receiver (IEQ-HR)

| | | | Receiver | | | | | |
|---|---|---|---|---|---|---|---|---|
| | | | HO #1 | | Receiver | | Receiver | |
| | | | Total | | HO #2 | | HO #3 | |
| | | Total # | # of | | Total # | | Total # | |
| | Weights | of IUs | IUs | % IUs | of IUs | % IUs | of IUs | % IUs |
| IU Category | (W_{T)} | available | Shared | Shared | Shared | Shared | Shared | Shared |
| B. Dept./Unit Specific | 3.0 | 5 | 3 | 60% | 2 | 40% | 1 | 20% |
| Information Units | | | | | | | | |
| C. Patient Specific | 7.0 | 5 | 5 | 100% | 5 | 100% | 5 | 100% |
| Information Units | | | | | | | | |
| (e.g. clinic condition) | | | | | | | | |
| Weighted Average | | | | 88% | | 82% | | 76% |
| Percentage | | | | | | | | |
| IEQ-HR | | | | 8.800 | | 8.200 | | 7.600 |

Interaction between the handoff sender and receiver may be evaluated as an interaction score (INT). Synchronous patient handoffs are better than asynchronous handoffs since the former allow for the opportunity to ask questions. They also allow for the opportunity to discuss difficult clinical situations, and allow for education of both the handoff sender and receiver. Thus, the valuations of synchronous v. asynchronous handoffs may be different. In this example, asynchronous patient handoff is weighted with a normal weight (W_{T}=1). Synchronous patient handoff is weighted twice as more important than the asynchronous patient handoff (W_{T}=2).

Patient handoff efficiency may be determined using a handoff length of time (HOLT) valuation. A HOLT may be basically a measure of time spent by the handoff sender and the handoff receiver in the process of preparing for and conducting the patient handoff. HOLT calculation start and stop times may vary depending on the nature of the handoffs. HOLTS may be represented as integer values of 3, 2 or 1 depending if the time is within one standard deviation of mean or two SD or three SD, respectively. The mean handoff time maybe calculated in real time as more patient handoffs occur. Synchronous patient handoff time is calculated from the start of the patient handoff by the handoff sender till the handoff completion time by the handoff receiver. Asynchronous patient handoff time primarily comprises two elements i) start and stop time of the handoff sender and ii) start and stop time of the handoff receiver. Asynchronous patient handoff time can also comprise the length of time spent by the handoff sender to complete/review patient profile(s) in the application (e.g. FIG. 4B 455-465). Some examples of other HOLT calculations examples for different kinds of patient handoffs: Patient Transfer from Emergency Department by an ED Nurse to Inpatient to an inpatient Nurse: ED Nurse Transfer Start Time to ED Nurse Transfer Stop Time+Inpatient Nurse Transfer Accept Start Time to Inpatient Nurse Transfer Accept Stop Time. Patient Handoff between two Hospitalists: Time spent on Reviewing/Completing Patient profiles by the Hospitalist Sender. This can happen over many days. Patient Handoff between two Residents: Time at the Start of Patient Handoff with the first patient by the Resident Sender to Time at the End of Patient Handoff with the last patient by the Resident Sender. Resident Receiver's time is not comprised in this HOLT. Some additional efficiency parameters may include the time difference between stop time of handoff sender and start time of handoff receiver especially for asynchronous handoffs, as well as interruptions: Any pause/delay longer than a pre-specified time in between the exchange of two information units is considered an interruption. Smaller the information unit lesser is the “pre-specified time” for interruption.

The AI module 590 described below may be used to help determine the weighting associated with categories of information units (e.g. patient specific IUs, department/unit specific IU) and weighting associated with standard deviations from mean in the handoff length of time (HOLT). In an embodiment, different weights and mapping to the handoff elements IEQ, INT, HOLT, and factors that help determine the patient handoff efficacy may be applied using AI.

Table 2 below helps demonstrate some examples of HOLT valuations for a synchronous and asynchronous handoff where the mean time for handoff in this example is seven minutes (HOLT values of 3, 2, and 1 may be determined if the handoff time is within one standard deviation of mean or two SD of mean or three SD of mean. The mean may be calculated in real time as more patient handoffs occur):

TABLE 2

| | | | | | | Time from | | |
|---|---|---|---|---|---|---|---|---|
| | | | | | | Handoff | | |
| | | | | | | Sender | | |
| | | | | | | Stop Time | | |
| | Handoff | Handoff | Handoff | | | to | | |
| | Sender | Sender | Receive | Handoff | Handoff | Handoff | | |
| | Start | Stop | Start | Receiver | Length | Receiver | | HOLT |
| | Time | Time | Time | Stop Time | of Time | Start Time | | Value |
| Sync- | 19:11 | 19:15 | 19:15 | 19:17 | 6 min | <1 | min | 3 |
| HO | | | | | | | | |
| Async- | 19:11 | 19:15 | 07:23 | 07:27 | 8 min | 12:08 | Hr | 3 |
| HO | | | | | | | | |

Using the equation provided above, the following examples from tables 1-1, 1-2, and table 2 data may be used to illustrate a sample PHS calculation. For a synchronous handoff using sender and receiver handoff #1 and a HOLT value of 3:

PHS=(IEQ-HS+IEQ-HR)/2×INT×HOLT

PHS=(9.667+8.8)/2×2×3=55.401

For an asynchronous handoff using sender and receiver handoff #2 with a HOLT value of 2:

PHS=(9.242+8.2)/2×1×2=17.442

A composite patient handoff quality measurement may be determined and reported. The composite Patient Handoff Index (PHI) maybe be determined by aggregating patient handoff score across a department or a unit or an institution. To calculate the PHI, the sum of the patient handoff scores of patient handoffs in a department or unit or an institution may be divided by a divisor, the Handoff Divisor (hd). The divisor may be adjusted based on multiple risk criteria such as the size of the patient population in a department or an institution, or the complexity of patient population (a tertiary care center might be expected to have a higher proportion of sicker or more complex patients or patients with higher prevalence of comorbidities versus patients in a community hospital) or the training of healthcare providers (a higher proportion of specialized health care providers might be expected to have a better plan of care, or better diagnosis or better handoffs leading to better patient outcomes) and the like. In its simplest form the handoff divisor may be composed of the number of patient handoffs in a department or a unit or an institution making the PHI a simple arithmetic average.

PHI=ΣPHS/hd
where PHS is the individual handoff score and hd is the handoff divisor.

The handoff divisor may be adjusted to compare patient handoff efficacy across departments or units or institutions. For example, if the HOLT is more than two standard deviations for a handoff, in a patient with high complexity, the HOLT value will be 1 to calculate the PHS. In this scenario the handoff divisor may adjust or normalize the PHS given that the patient complexity may be high, for example, as in a patient in the intensive care unit (ICU). So, if an institution has higher number of ICU patients versus another institution the composite patient handoff quality, the PHI, may be adjusted or normalized for HOLT. The handoff divisor may be adjusted or normalized if the patient handoff senders and/or receivers have different levels of training. For example, if an institution has a higher proportion of certified nursing assistants as opposed to registered nurses performing patient handoffs, reflecting different IEQ-HS and IEQ-HR values, then the handoff divisor may adjust or normalize the PHS.

For departments or units or institutions with small number of patient handoffs in a reference time period, hierarchical regression models may be used to reduce the chances the PHI may fluctuate wildly from one reference time period to the next or that these departments or units or institutions may be incorrectly labeled with lower or higher PHI.

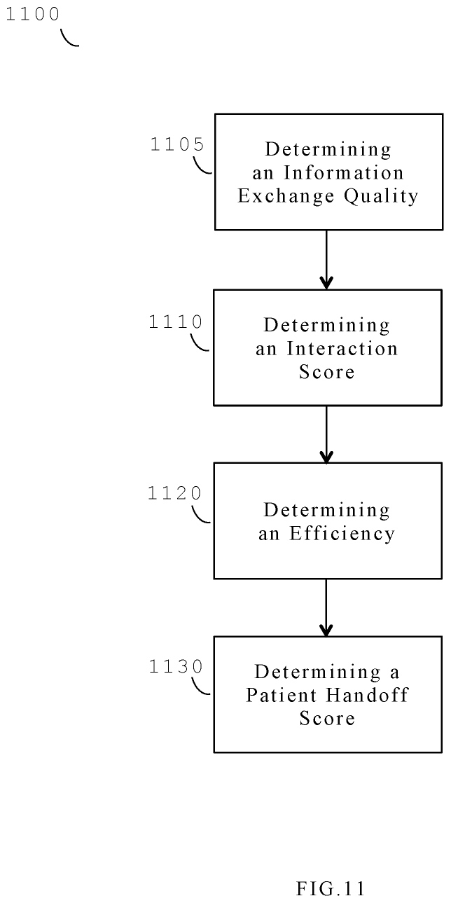

FIG. 11 illustrates a method 1100 or determining a patient handoff score (PHS). The method may use the formulas and methods described above. At 1105, determining a IEQ. The IEQ may be determined for the sender (IEQ-HS) or the handoff receiver (IEQ-HR). At 1110, determining an interaction value (INT). At 1120, determining the efficiency of the handoff. At 1130 determining a patient handoff score (PHS) based on the combination of IEQ-HS, IEQ-HR, INT, and efficiency of the handoff from steps 1105, 1110, and 1120.

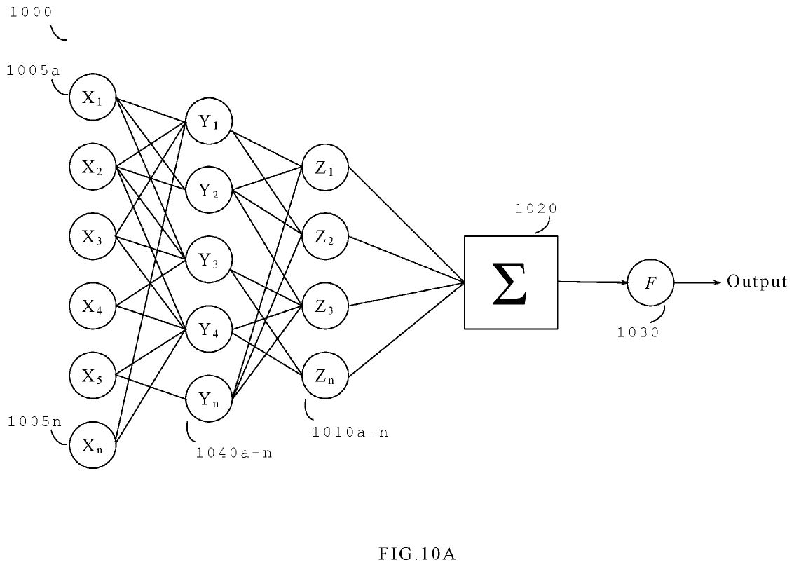

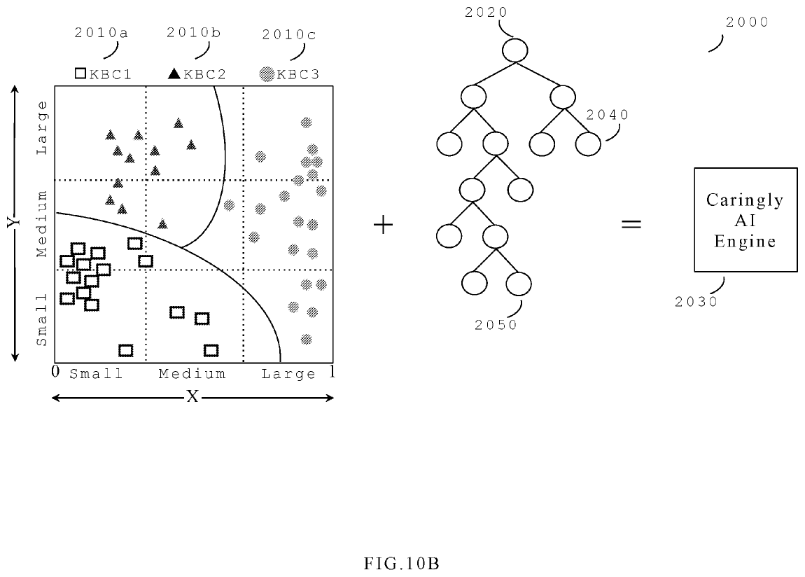

FIG. 10A illustrates a method 1000 of using AI in an embodiment. AI may be used to help model information units (modify and create), help create and modify triggers and actions, help parse and transform information, help prioritize information, and improve the embodiments by learning. The AI in the described embodiments, may improve the medical field. Risk may be deeply embedded in the practice of medicine. The outcomes of which may have dramatic impacts on patients' lives. Moreover, lack of transparency that arises from AI systems that predict and make recommendations without exposing the underlying reasons may be highly problematic in healthcare. The AI systems that employ machine learning over large sets of data and then map end user specific features into a class to predict traits or make recommendations about the end user without exposing the underlying reasons may be hiding biases in the algorithms about human prejudices and artifacts hidden in the training data. For example, an existing solution may recommend that a patient be given medication to raise their blood pressure, because it's low; but fail to let the caregiver understand where that recommendation is coming from. It could be that the patient was given prior medication that inadvertently lowered their blood pressure, but that specific information wasn't told to a receiving nurse by the sending nurse during handoff. It could be that the sending nurse didn't even known about the medicine's interaction to mention it (e.g. lack of skill, lack of information, etc.). An AI that could detect the error in the first place and guide the caregivers during a handoff is contemplated in the embodiments, as well as, an AI that could provide the users with an explanation of how it arrived at the recommendation. AI in an embodiment may aim to mitigate these risks by employing transparent/explainable AI. The end-users may receive a prediction of a certain outcome with an explanation for the underlying reasons and may also provide a recommendation based on that underlying reason.

In FIG. 10.A a few hidden layers and a few hidden nodes (e.g. 1040a-n) are shown for simplicity, but deep learning (multiple hidden layers and multiple nodes) may be used. Data inputs that may be sourced into the AI as inputs 1005a-n may come from an identification module 510, a memory/databases module 501, an administration module 530, audit log/time tracker module 561, a workflow module 550m a patient information module 505, a handoff type module, a clinical information module, a scoring/reporting module 595, a customization module 585, a learning/feedback module 591, a “tell” and “ask” assist module, or as described in more detail below, a KB classifiers database. Biased nodes may also provide an input source. The weights 1010a-n (modeling and mapping) may be attributed to each corresponding input. The summation of the weights 1020 may then be applied and sent to a step, sigmoid, rectified linear, or softmax function 1030 to provide an output. AI is a newly developing technology and future created functions may be available for use and operate as an equivalent to the functions listed herein. As stated above, the output of 1030 may be input to other hidden nodes not shown. The AI model may use supervised learning, unsupervised learning, reinforced learning or a combination of them for classifying and grouping data. For example, with supervised learning, data from the clinical information module 540 may be labeled: diagnostics, lab results, allergies, and so forth. The initial training and weights applied with supervised learning may be provided as a starting point by the application. For example, the AI may be trained to classify all the “labeled” lab results with a given weight if the results are outside of a typical range. Or the AI may use existing or template IUs for initial training. The AI may use information from the feedback or learning module 591 and information sources to modify the weights (mapping/modeling) to improve the embodiments. With unsupervised learning, more hidden nodes would be provided to cluster unlabeled inputs and detect the similarities or anomalies between them to form clusters or patterns. For example, the AI may analyze documents provided by the information sources and learn to know that certain documents are x-ray imaging results and create it's own classification for them.

FIG. 10B illustrates another method 2000 of using AI in an embodiment. FIG. 10B illustrates an expert knowledge base (KB) based classifiers (based on fuzzy rules), representing both a combination of domain ontology and expert's knowledge. Domain ontologies may be a semantic web language designed to represent rich and complex knowledge about things, groups of things, and relations between things. For example, the domain ontology may be encoded in the web ontology language (OWL). OWL, a semantic markup language based on the description logic, is used to develop semantic web applications, whereas domain knowledge may be represented as a set of concepts with individuals involved, their properties and the relationship between these concepts. The domain ontology may consist of at least two main categories: i) concepts related to the patient variables (i.e. clinical signs, symptoms, test and imaging results, pathologies, blood lab results, etc.) and ii) variables related to the clinical management (i.e. the plan of care objectives, treatment, goals, medical actions). However, more than two categories of domain ontology may be used. This may be specifically used to enrich the description of patient care as a function of patient handoff and medical actions at different levels of abstraction. This may be built in an iterative process including the use of existing ontologies. The knowledge base may be a medical information set compiled as IF-THEN rules obtained from domain experts and literature search. The knowledge base may build on existing IF-THEN rules for patient care in inpatient settings and may enrich and focus on patient handoffs to help mitigate the errors associated with handoffs. The KB based classifiers 2010a, 2010b, 2010c may work by classifying units of knowledge by degree of interpretability in two dimensions X and Y with a score between 0 (lowest) and 1 (highest). In an embodiment, a high level may be a value between 0.6 and 1.0., a low value may be a value between 0.0 and 0.3, and a medium value may be a value between 0.31 and 5.9. The values ranges may be determined differently in other embodiments. Three KB classifiers 2010a-c are shown, but at least one KB classifiers are envisioned in the embodiments. Thus, there can be any number of KB classifiers. KB classifiers are meant to compile into sets related medical knowledge. For example, one KB set may deal with respiratory related medical issues compiled from domain ontology and expert knowledge. The degree of interpretability may be determined in reference to a human curated dataset exemplifying the ideal and optimal interpretation and represents the target for the design. The higher the interpretability of a machine learning model, the easier it may be for someone to comprehend why certain decisions or predictions have been made. A model may be better interpretable than another model if its decisions are easier for a human to comprehend than decisions from the other model. The interpretability may further have a coarse-grained representation i.e. small (low), medium and large (or high). So for example, the Y-axis may represent the domain ontology category of patient variables (arbitrarily illustrated as the Y-axis in FIG. 10B), and the X-axis may represent domain ontology of clinical management variables (arbitrarily illustrated as the X-axis in FIG. 10B). So as an example, the “large” values plotted on the Y-axis may represent twenty blood test results from a patient, while the “small” values plotted on the Y-axis may represent three blood test results from a patient. The X-axis “large” value plotted may represent the number of times a patient was turned over during a twenty-four hour period, while a “small” value plotted on the X-axis may represent that no turnover was provided (or it's unknown). Thus, the highest level of interpretability will be in the top right corner of the X, Y chart. Classifiers exhibiting high interpretability in both dimensions may preferentially be selected and may later be combined with fuzzy decision tree 2020 (FIG. 10b) to create the “Caringly” AI engine 2030. Another way of stating this may be to say that the KB classifiers selected (data sets) may be used as input to nodes of tree 2020. The fuzzy decision tree 2020 is a collection of decision nodes at different levels and the leaves (final last node) represents the result achieved when all attributes of the subject are used on the path. Walking the tree from top to bottom represents an increasing level of specificity as more attributes are evaluated. During this walk, if the classification in the succeeding layer results in reduced specificity then that path is abandoned and the decision tree is navigated recursively with varying attributes to reach a leaf. For example, a KB classifier is selected and provided as input to a tree 2020. There may be multiple trees and a recursive analysis performed. The first branches of the tree 2020, for example, may decide the pathology. The next set of nodes may decide renal disease. If the KB classifiers where from a “small” or lower level of interpretability, then it's possible that the decision tree would stop short on leaf 2040. For example, a blood test was missing from the data set that would have allowed the tree to decide to move forward. Leaf 2040 may have a lower value of predictability associated with it. For example, it may have a 65% predictability score. However, if the KB classifiers had a “large” level of interpretability, then the decision tree could move down the nodes to decide, renal failure, finally at leaf 2050 severe renal failure. Leaf 2050 may have a 95% predictability score. This interpretable AI engine may represent machine interpretable knowledge base along with the decision tree/s , and may be added to AI/ML classifiers (FIG. 10A, FIG. 5 505 & 590). This may provide a solid basis for interpretability of output (decision). For a given instance of data (clinical+handoff) the explainable system's decision-making process may go through the process of aggregating all outputs and selecting the one which matches at least one interpretable classifier. The selected output may be then converted into linguistic descriptions (understandable by humans) formulating both the explanation as well as the recommendation. For example, the Caringly AI embodiment might predict that the risk of complications from a hospital associated pressure injury (HAPI) in a patient ‘John Smith’ who spent five days in a hospital might be 65% based on a complicated set of inputs and models. The explanation may then highlight each relevant input and explain the input weights/decisions in terms a human may understand: “the patient John Smith has been confined to bed for the last five days, with completely limited sensory perception, completely limited mobility, constantly moist skin, very poor nutrition, and limited turnover in the bed. Six of the ten criteria match and hence the confidence level in HAPI risk may be 65%”. The recommendation may be for the patient not to be discharged home but moved to a skilled nursing facility. The explanation may also provide the inputs that were not used and why they were not considered important etc. In an embodiment, whether creating KB classifiers or creating fuzzy decision trees or determining the degree of interpretability, the trained AI models performance may be measured in terms of specificity, accuracy and sensitivity. The performance measurements may be defined by:

Specificity=TN/(TN+FP)

Accuracy=(TP+TN)/(TP+TN+FP+FN)

Sensitivity=TP/(TP+FN)
where, TP and TN are number of true positives and true negatives, respectively.
FP and FN are the number of false positives and false negatives, respectively.

In an embodiment a method for patient care handoff, is described comprising initiating a handoff; accepting the handoff invitation; facilitating the handoff, generating at least one patient information unit (IU) to be exchanged for handoff, and utilizing a tell and ask assist, the tell and ask assists utilizing the at least one patient information unit (IU); accepting the handoff; and completing the handoff. Wherein the generating at least one patient information unit (IU) comprises: receiving information; parsing information; transforming information; and prioritizing information. Further comprising: creating a checklist. Further comprising: using artificial intelligence (AI) to modify and create an information unit (IU).

In another embodiment, a method for patient care handoff, is described comprising:

initiating a handoff; selecting a receiver; facilitating the handoff, generating at least one patient information unit (IU) to be exchanged for handoff, and utilizing a tell and ask assist, the tell and ask assists utilizing the at least one patient information unit (IU); accessing the handoff; accepting the handoff; and completing the handoff. Wherein the generating at least one patient information unit (IU) comprises: receiving information; parsing information; transforming information; and prioritizing information. Further comprising: using artificial intelligence (AI) to modify and create an information unit (IU). Further comprising: creating a checklist.

In an embodiment, a system for patient handoff, is described, comprising

a server comprising an application control module, a patient information module,

a clinical information module comprising, an IU module comprising code to generate IUs, an engine module comprising, a tell and ask module, and a dashboard module; a mobile device, the server allowing access to the dashboard via the mobile device. Further comprising a customization module comprising. Further comprising, a client application module. Further comprising, a scoring module. Wherein the engine module, comprising: a priority module comprising, a parsing module, a transforming module, a triggers and actions module, and an AI module.

In an embodiment, a system for patient care handoff, is described, comprising:

a server configured to communicate with a sending mobile device and a receiving mobile device; the server configured to facilitate a handoff between the sender mobile device and receiver mobile device. The server comprising an application control module, a patient information module, a clinical information module comprising, an IU module comprising code to generate IUs, an engine module comprising, a tell and ask module, and a dashboard module.

A method of processing information for patient care handoff, comprising:

inputting input data of administration information, audit log and time tracker information, workflow information, patient information, handoff type information, clinical information, sender profiles, receiver profiles, device types, entity profiles, learning information, modeling the input data in a plurality of engines modules within a computing environment, the engine modules executed by an application control module, the engine models configured to create or modify information units (IU) by providing a template IU; Predicting an IU by parsing, transforming, and prioritizing patient information; comparing the template IU to the predicted IU to identify relationships between the predicted and template IUs; creating and generating as an output an IU.

A method of processing information for patient care handoff, comprising:

inputting input data from at least one knowledge base (KB) based classifiers; selecting at least one classifier with a high level of interpretability between at least two ontology domain categories; running the selected classifier through at least one decision tree; generating an interpretable AI engine based on the selected at least one classifier that was ran through the at least one decision tree; and outputting AI classifiers. The method further comprising, aggregating all AI classifier outputs and selecting the one that matches at least one interpretable classifier; converting the selected output into linguistic descriptions; and formulating the explanation as well as the recommendation.

In other embodiments, the processing modules may be implemented using a shared processing device, individual processing devices, or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions.

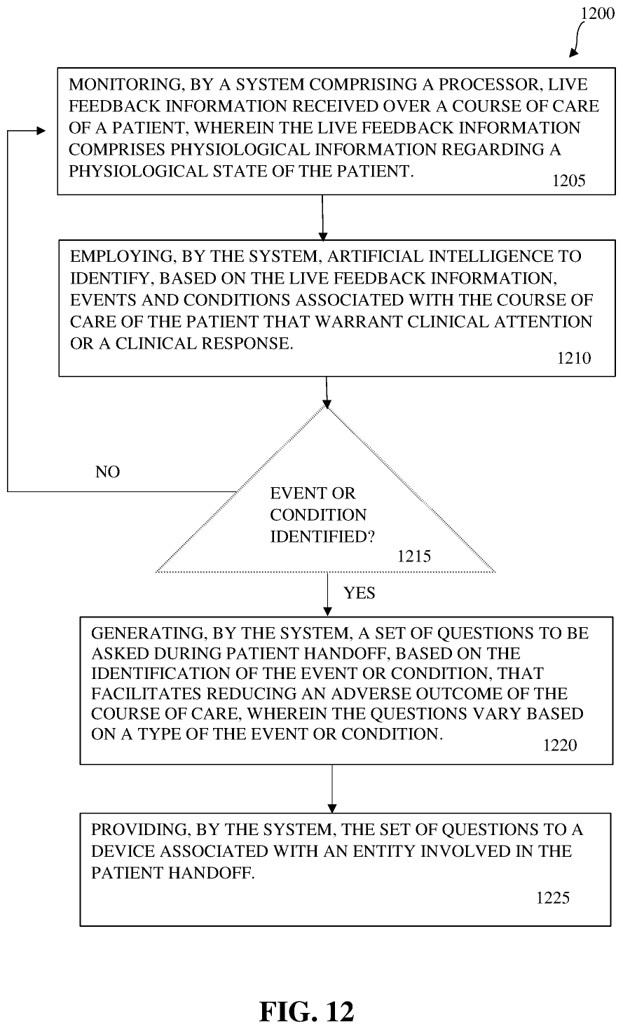

According to another embodiment illustrated in FIG. 12, a computer implemented method 1200 comprises continuously monitoring live feedback received over a course of care of a patient, wherein the live feedback comprises physiological information regarding a physiological state of the patient 1205. The method further comprises employing AI to identify, based on the live feedback information, an event or condition associated with the course of care of the patient that warrants clinical attention 1210. The method further comprises a conditional operation to determine whether an event or condition has been identified 1215. If yes, the method proceeds to generating questions related to the identified event or condition. If no, the method loops back to monitoring live feedback. The method further comprises generating questions for use during patient handoff, based on the identification of the event or condition, that facilitates reducing an adverse outcome or other mistake during the course of care, wherein the questions vary based on a type of the event or condition 1220, and providing the questions to a device associated with an entity involved in the patient handoff 1225.

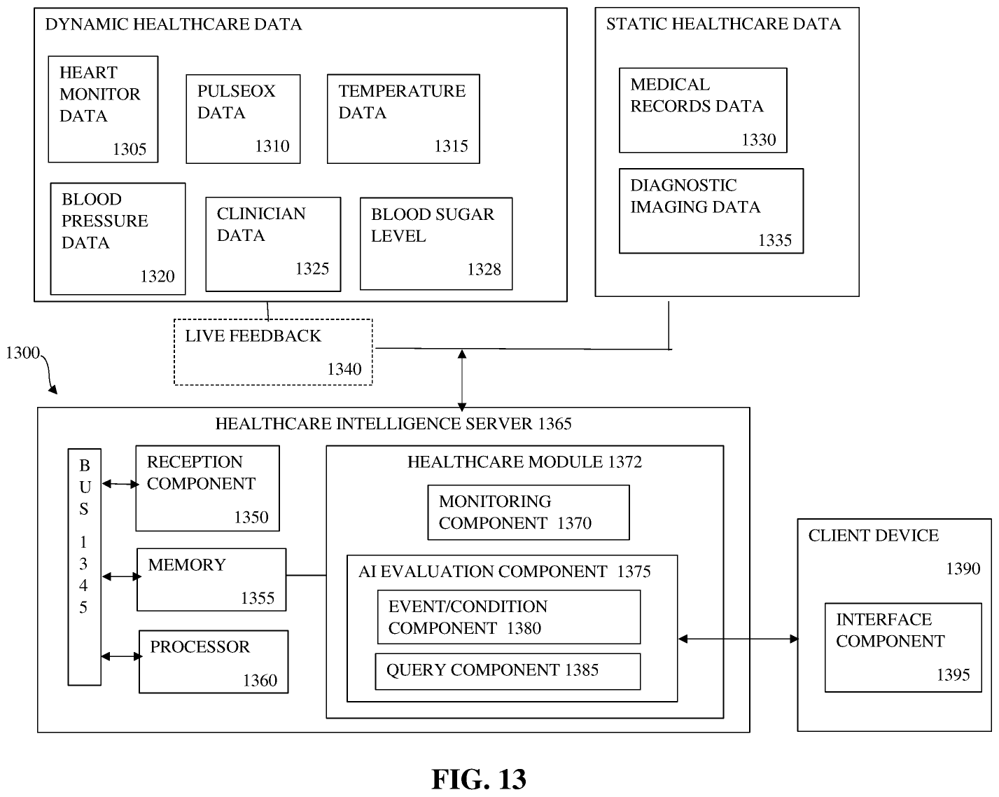

In one embodiment shown in FIG. 13, a system 1300 is provided that comprises a memory 1355 that stores computer executable components and a processor 1360 that executes the computer executable components stored in the memory. The computer executable components comprise a monitoring component 1370 configured to monitor live feedback 1345 received over a course of care of a patient, wherein the live feedback comprises physiological information regarding a physiological state of the patient, and an event/condition identification component 1380 configured to employ AI to identify, based on the live feedback information, an event or condition associated with the course of care of the patient that warrants clinical attention. The physiological information may be dynamic data acquired from a clinician and/or various diagnostic medical devices such as a remote telemetry heart monitor and pulse oximeter, EKG, EEG, blood pressure monitor, blood sugar monitor, thermometer, etc. The physiological information may also include static healthcare data acquired from a patient's medical records, diagnostic imaging data (e.g., X-rays, MRI, CT) or other medical reports. A reception component 1350 such as an antenna, network interface card, modem, Bluetooth® receiver, RFID receiver, and the like is configured to receive the live feedback information which includes the dynamic healthcare data, the static healthcare data and data from other sources such as client device 1390. The computer executable components further comprise a query component 1385 configured to determine a set of questions to ask during handoff, based on the identification of the event or condition, that facilitates reducing an adverse outcome during the course of care, wherein the questions vary based on a type of the event or condition, and wherein the query component is further configured to generate the questions and provide the questions to a device 1390 associated with an entity involved in the patient handoff process.

In some implementations, the event/condition identification component can be configured to identify the event or condition using machine learning analysis of historical healthcare delivery information regarding same or similar courses of care and one or more standard operating procedures defined for the course of care. The event/condition identification component can further be configured to identify the event or condition based on information regarding a medical health history of the patient.

In one embodiment, the computer executable components include a notification component configured to generate and transmit a notification regarding the event or condition to the device. The notification can comprise a mechanism for providing an acknowledgment indicating the notification was received.

In another implementation, the client device comprises an interface component 1395 and wherein questions generated by the query component are updated based on information received from the interface component in real-time to reflect interactions between participants in the handoff process, wherein the interface component records and processes verbal interactions between handoff participants using natural language processing. The verbal interactions are provided to the AI evaluation component for interpretation. The query component is then updated with new questions based on the interpreted verbal interactions in real-time. With this implementation, the system can further comprise a monitoring parameter update component configured to determine one or more parameters related to the event or condition, and wherein the query component is updated to generate questions designed to acquire information related to the one or more parameters. The system can further comprise a prioritization component configured to determine a priority order of the events and conditions and wherein the query component is updated to reflect the priority order.

In some embodiments, elements described in connection with the disclosed systems can be embodied in different forms such as a computer-implemented method, a computer program product, or another form.

The described embodiments or any part(s) or function(s) thereof, may be implemented using hardware, software, or a combination thereof, and may be implemented in one or more computer systems or other processing systems. A computer system for performing the operations of the described embodiments and capable of carrying out the functionality described herein can comprise one or more processors connected to a communications infrastructure (e.g., a communications bus, a cross-over bar, or a network). Various software embodiments are described in terms of such an exemplary computer system. After reading this description, it will become apparent to a person skilled in the relevant art(s) how to implement the embodiments using other computer systems and/or architectures.

The foregoing description of the preferred embodiments has been presented for purposes of illustration and description. It is not intended to be exhaustive or to limit the embodiments to the precise form or to exemplary embodiments disclosed. Obviously, many modifications and variations will be apparent to practitioners skilled in this art. Similarly, any process steps described might be interchangeable with other steps in order to achieve the same result. The embodiments were chosen and described in order to best explain the principles of the embodiments and its best mode practical application, thereby to enable others skilled in the art to understand the various embodiments and with various modifications as are suited to the particular use or implementation contemplated. It is intended that the scope of the embodiments be defined by the claims appended hereto and their equivalents. Reference to an element in the singular is not intended to mean “one and only one” unless explicitly so stated, but rather means “one or more.” Moreover, no element, component, nor method step in the described disclosure is intended to be dedicated to the public regardless of whether the element, component, or method step is explicitly recited in the following claims. No claim element herein is to be construed under the provisions of 35 U.S.C. Sec. 112, sixth paragraph, unless the element is expressly recited using the phrase “means for . . . .”

In addition, the conjunction “and” when used in the claims is meant to be interpreted as follows: “X, Y and Z” means it can be either X , Y or Z individually, or it can be both X and Y together, both X and Z together, both Y and Z together, or all of X, Y, and Z together.

It should be understood that the figures illustrated in the attachments, which highlight the functionality and advantages of the described embodiments, are presented for example purposes only. The architecture of the described embodiments are sufficiently flexible and configurable, such that it may be utilized (and navigated) in ways other than that shown in the accompanying figures.

Furthermore, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the described embodiments in any way. It is also to be understood that the steps and processes recited in the claims need not be performed in the order presented.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function. A process or method may be implemented with a processor, or similar device, or any combination of hardware and software.

Moreover, a storage medium may represent one or more devices for storing information, including read-only memory (ROM), random access memory (RAM), magnetic disk storage mediums, optical storage mediums, flash memory devices and/or other machine-readable mediums, processor-readable mediums, and/or computer-readable mediums for storing information. The terms “machine-readable medium”, “computer-readable medium”, and/or “processor-readable medium” may comprise, but are not limited to non-transitory mediums such as portable or fixed storage devices, optical storage devices, and various other mediums capable of storing, containing or carrying instruction(s) and/or information. Thus, the various methods described herein may be fully or partially implemented by instructions and/or information that may be stored in a “machine-readable medium”, “computer-readable medium”, and/or “processor-readable medium” and executed by one or more processors, machines and/or devices. Moreover, a micro processor, or similar device may have internal or external memory associated with it.

The various features of the embodiments described herein can be implemented in different systems without departing from the embodiments. It should be noted that the foregoing embodiments are merely examples and are not to be construed as limiting the embodiments. The description of the embodiments is intended to be illustrative, and not to limit the scope of the claims. As such, the described teachings can be readily applied to other types of apparatuses and many alternatives, modifications, and variations will be apparent to those skilled in the art.

What is claimed is:

1. A system for improving patient handoff, the system comprising:
a memory that stores computer executable components;
a processor that executes the computer executable components stored in the memory, wherein the computer executable components comprise:
a monitoring component configured to monitor live feedback information received over a course of care of a patient, wherein the live feedback comprises physiological information regarding a physiological state of the patient;
an event or condition identification component configured to employ artificial intelligence to identify, based on the live feedback information, an event or condition associated with the course of care of the patient that warrants clinical attention or a clinical response; and
a query component configured to determine a set of questions designed to elicit relevant information about the patient during the patient handoff, based on the identification of the event or condition, that facilitates reducing an adverse outcome during the course of care, wherein the questions vary based on a type of the event or condition, and
wherein the query component is further configured to generate and provide the questions to a device associated with an entity involved in the patient handoff.

2. The system of claim 1. further comprising:
an information unit (IU) module comprising code to generate and modify IUs;
a priority module comprising code to prioritize patient information;
a parsing module comprising code to parse information;
a transforming module comprising code to transform information;
a learning module; and
a triggers and actions module comprising code to create, modify, and execute application triggers and actions.

3. The system of claim 2, further comprising:
a scoring module comprising code to measure the quality of the handoff.

4. The system of claim 3, wherein the scoring module comprises code to determine a patient handoff score (PHS), the patient handoff score (PHS) based on the combination of:
a numeric value representing a nature of the information exchanged quality (IEQ) for the handoff, a numeric value representing an interaction value (INT) between a handoff sender and a receiver; and a numeric value representing an efficiency of the handoff.

5. The system of claim 4, wherein the patient handoff score (PHS) is represented as a numeral value between zero and sixty determined by the formula (IEQ-HS+IEQ-HR)/2×INT×HOLT.

6. The system of claim 5, wherein the numeric value representing a nature of the information exchanged quality (IEQ) for the handoff sender (IEQ-HS) and the handoff receiver (IEQ-HR) is determined by attributing a weight to an information unit (IU) category, determining the total number of IUs available per weighted category for the handoff entity, determining the total number of IUs shared per weighted category for the handoff entity, determining the percentage of the IUs shared to the total number of IUs available per weighted category for the handoff entity, determining a weighted average percentage for all of the weighted IU categories percentages of IUs shared to the total number of IUs available for the handoff entity, and representing IEQ-HS and IEQ-HR numerically as the weighted average percentage for the entity divided by ten.

7. The system of claim 6, wherein the interaction value (INT) is weighted differently for a synchronous handoff than an asynchronous handoff.

8. The system of claim 6, The application of claim 6, wherein the numeric value representing the efficiency of the handoff is based on a handoff length of time (HOLT).

9. The system of claim 8, wherein the HOLT is determined differently for different types of handoffs.

10. The system of claim 8, wherein the HOLT for a synchronous patient handoff is calculated from the start of the patient handoff by the handoff sender till the handoff completion time by the handoff receiver; and
the HOLT for an asynchronous patient handoff time comprises the start and stop time of the handoff sender and the start and stop time of the handoff receiver.

11. A method for improving a patient handoff, the method comprising:
using a processor to execute the following computer executable instructions stored in a memory to perform the following acts:
monitoring live feedback information received over a course of care of a patient, wherein the live feedback information comprises physiological information regarding a physiological state of the patient;
employing artificial intelligence to identify, based on the live feedback information, an event or condition associated with the course of care of the patient that warrants clinical attention or a clinical response;
generating a set of questions for use during the patient handoff, based on the identification of the event or condition, that facilitates reducing an adverse outcome during the course of care, wherein the questions vary based on a type of the event or condition; and
providing the questions to a device associated with an entity involved in the patient handoff.

12. The method of claim 12, further comprising:
determining a patient handoff score (PHS), the patient handoff score (PHS) based on the combination of:
determining a numeric value representing a nature of the information exchanged quality (IEQ) for the handoff;
determining a numeric value representing an interaction value (INT) between a handoff sender and a receiver; and
determining a numeric value representing an efficiency of the handoff.

13. The method of claim 13, wherein the patient handoff score (PHS) is represented as a numeral value between zero and sixty determined by the formula (IEQ-HS+IEQ-HR)/2×INT×HOLT.

14. The method of claim 14, wherein the numeric value representing a nature of the information exchanged quality for the handoff sender (IEQ-HS) and the handoff receiver (IEQ-HR) is determined comprising:
attributing a weight to an information unit (IU) category;
determining the total number of IUs available per weighted category for the handoff entity;
determining the total number of IUs shared per weighted category for the handoff entity;
determining the percentage of the IUs shared to the total number of IUs available per weighted category for the handoff entity;
determining a weighted average percentage for all of the weighted IU categories percentages of IUs shared to the total number of IUs available for the handoff entity;
and representing IEQ-HS and IEQ-HR numerically as the weighted average percentage for the entity divided by ten.

15. The method of claim 14, wherein the interaction value (INT) is weighted differently for a synchronous handoff than an asynchronous handoff.

16. The method of claim 14, wherein the numeric value representing the efficiency of the handoff is based on a handoff length of time (HOLT) and is determined differently for different types of handoffs.

17. The method of claim 16, wherein the HOLT for a synchronous patient handoff is calculated from the start of the patient handoff by the handoff sender till the handoff completion time by the handoff receiver;
the HOLT for an asynchronous patient handoff time comprises the start and stop time of the handoff sender and the start and stop time of the handoff receiver.

18. The method of claim 12, further comprising:
generating an information unit (IU) comprising:
receiving patient information;
transforming patient information; prioritizing patient information; and
using artificial intelligence (AI) to modify and create the information unit (IU).

19. The method of claim 12, further comprising:
calculating a patient handoff index (PHI) by determining a sum of the patient handoff scores (PHS) and dividing the sum by a divisor (hd).
